(19) United States
(12) Patent Application Publication
(10) Pub. No.: US 2016/0106883 A1
(43) Pub. Date: Apr. 21, 2016

(54) Processes For Mixing Fibrinogen and Thrombin Under Conditions That Minimize Fibrin Formation While Preserving Fibrin-forming Ability, Compositions Produced by These Processes, and the Use Thereof

(71) Applicant: STB, Ltd., Bellevue, WA (US)

(72) Inventors: Martin MacPhee, Darnestown, MD (US); Jerry Kanellos, Eltham (AU); Belinda Wilmer, Martinsburg, WV (US); Dawson Beall, Germantown, MD (US); Shirley Miekka, Colorado Springs, CO (US)

(21) Appl. No.: 14/884,333

(22) Filed: Oct. 15, 2015

Related U.S. Application Data
(60) Provisional application No. 62/064,291, filed on Oct. 15, 2014.

(51) Int. Cl. A61L 26/00

(52) U.S. Cl. CPC A61L 26/0047; A61L 26/0042; A61L 2400/04; A61L 2300/60; A61L 2300/45; A61L 2300/418; A61L 2300/254; A61L 2300/252

(57) ABSTRACT

Fibrin Sealant products are used for topical hemostasis and tissue adherence. They are composed of two main reagents, fibrinogen and thrombin. When mixed in solution fibrinogen is converted to fibrin upon the addition of activated thrombin. Therefore typically these two components are stored separately in a lyophilized or liquid state, and mixed, upon or immediately before, application to a patient. While effective, these products require significant preparation that must take place immediately before application, thus delaying treatment and limiting the use of these haemostatic products to the treatment of mild forms of low pressure and low volume bleeding. Attempts to eliminate this delay and expand the usefulness and effectiveness of these products have resulted in products produced by processes that require the separation of these components and their deposition in distinct layers within the product. The processes described herein permit the mixing of fibrinogen and thrombin during product manufacture, without excessive fibrin formation. The resulting ‘pre-mixed’ fibrin sealant material can then be stored in either a frozen or dried state, or suspended in a non-aqueous environment. Activation of the material to form therapeutic fibrin sealant is accomplished by permitting the product to thaw (if frozen) or by the addition of water or other aqueous fluid, including blood, or other bodily fluids, if dried or suspended in a non-aqueous environment. The resulting material can be used to make a product in which a pre-mixed form of activatable fibrin sealant is a desired component.

### I. FIELD OF THE INVENTION

This invention relates to processes for the mixing of fibrinogen with thrombin under conditions that limit their interaction to form fibrin, until that interaction is desired. An application for such a process would be in the manufacturing of a fibrin sealant-based haemostatic dressing where the fibrinogen and thrombin mixture would not generate significant levels of fibrin until it is desired that they do so, such as when the dressing is applied to wounded tissue. Such products could have differing fibrinogen/thrombin ratios, and differing ratios within a specific product, in order to maximize the efficacy of the product while minimizing its expense.

The invention also relates to compositions of mixtures containing fibrinogen and thrombin which have levels of fibrin that are sufficiently low so as to permit adequate conversion of fibrinogen to fibrin during application to the patient to ensure the effective use of the product.

The invention also relates to methods of treating a patient in need of therapy with a composition or product made by the processes described above.

### II. BACKGROUND OF THE INVENTION

The control of hemorrhage (bleeding) is a critical step in first aid and field trauma care. Unfortunately, the materials and methods available to stop bleeding in prehospital care (gauze dressings, direct pressure, and tourniquets) have not changed greatly in the past 2000 years. L. Zimmerman et al., Great Ideas in the History of Surgery (San Francisco, Calif.: Norman Publishing; 1993), 31. Even in good hands they are not uniformly effective, and the occurrence of excessive bleeding or fatal hemorrhage from an accessible site is not uncommon. J. M. Rocko et al., J. Trauma 22:635 (1982).

Mortality data from Vietnam indicates that 10% of combat deaths were due to uncontrolled extremity hemorrhage. SAS/STAT Users Guide, 4th ed. (Cary, N.C.: SAS Institute Inc.; 1990). Up to one third of the deaths from exsanguination during the Vietnam War could have been prevented by the use of effective field hemorrhage control methods. SAS/STAT Users Guide, 4th ed. (Cary, N.C.: SAS Institute Inc.; 1990).

Although civilian trauma mortality statistics do not provide exact numbers for prehospital deaths from extremity hemorrhage, case and anecdotal reports indicate similar occurrences. J. M. Rocko et al., J. Trauma 22:635 (1982). These data suggest that a substantial increase in survival can be effected by the prehospital use of a simple and effective method of hemorrhage control.

Liquid fibrin sealants have been used for years as an operating room adjunct for hemorrhage control. J. L. Garza et al., J. Trauma 30:512-513 (1990); H. B. Kram et al., J. Trauma 30:97-101(1990); M. G. Ochsner et al., J. Trauma 30:884-887 (1990); T. L. Matthew et al., Ann. Thorac. Surg. 50:40-44 (1990); H. Jakob et al., J. Vasc. Surg., 1:171-180 (1984). The first mention of tissue glue used for hemostasis dates back to 1909. Current Trends in Surgical Tissue Adhesives: Proceedings of the First International Symposium on Surgical Adhesives, M. J. MacPhee et al., eds. (Lancaster, Pa.: Technomic Publishing Co; 1995). The widespread use of fibrinogen and thrombin was common in the last year of World War II, but was abandoned because of the transmission of hepatitis. D. B. Kendrick, Blood Program in WW II (Washington. D.C.: Office of the Surgeon General, Department of Army; 1989). 363-368.

Currently, single donor fibrin sealants are widely used clinically, not only for hemorrhage control but in various surgical situations. (W. D. Spotnitz, Thromb. Haemost. 74:482-485 (1995); R. Lerner et al., J. Surg. Res. 48:165-181 (1990)). Even more extensive use is limited by the strict requirements for temperature control, availability of thawed blood components, and the need for mixing of components. Additional problems with the standard fibrin sealants stem from the transfusion risk of human cryoprecipitate (E. M. Soland et al., JAMA 274:1368-1373 (1995)), the low and variable amounts of fibrinogen in the cryoprecipitate (10-30 mg) (P. M. Ness et al., JAMA 241:1690-1691 (1979)), hypotensive responses to bovine thrombin (R. Berguer et al., J. Trauma 31:408-411 (1991)) and antibody responses to bovine thrombin (S. J. Rapaport et al., Am. J. Clin. Pathol. 97:84-91 (1992)).

The American Red Cross and others have developed plasma protein purification methods that seem to eliminate the hepatitis risk. R. F. Reiss et al., Trans. Med. Rev. 10:85-92 (1996). These products are presently being considered for approval by the Food and Drug Administration.

A dry fibrinogen-thrombin dressing (Tachocomb™, Hafslund Nycomed Pharma, Linz, Austria) is also available for operating room use in many European countries. U. Schiele et al., Clin. Materials 9:169-177 (1992). Present formulations of this dressing may use bovine thrombin. While this fibrinogen-thrombin dressing requires no premixing and is easy to use, its utility for field applications is limited by a requirement for storage at 4.degree. C. and the necessity for prewetting with saline solution prior to application to the wound.

Fibrinogen, thrombin and Factor XIII are 3 proteins that are part of the blood clotting cascade of animals. Briefly, when prothrombin is ‘activated’ to form thrombin, this cleaves off segments from fibrinogen which then self-polymerizes into a soluble fibrin polymer. Thrombin also activates Factor XIII to Factor XIIIa which then catalysis the cross-linking of the fibrin polymer to form a meshwork or net-like, insoluble structure. If the surrounding environment contains injured tissue, Factor XIIIa also crosslinks the fibrin to the tissue, sealing off injured tissue and blood vessels. Many products have been made using some or all of these proteins alone or in combinations with other ingredients (Tissue Sealants Available Today. MacPhee, M & Wilmer, K. in Tissue Glues In Cosmetic Surgery. Renato Saltz & Dean M. Toriumi, Eds. Quality Medical Publishing, Inc. 2004.), however all of these products rely upon maintaining a degree of separation between the reactants prior to application to the patient in order to prevent fibrin formation from proceeding prior to application to the patient's injured tissues. This is required because once fibrin has been fully crosslinked, it will no longer be bound to tissue by the action of Factor XIIIa, and the resulting product will have limited utility for hemostasis or the majority of additional desirable properties of fibrin sealants.

This constraint has limited the scope of inventions and applications for this material, as well as placing manufacturing constraints upon products that result in complex and/or expensive production processes, and producing products with sub-optimal characteristics.

Examples of these include the fibrin sealant-based wound dressings made by NycoMed and the American Red Cross (see U.S. Pat. Nos. 5,942,278; 6,762,336 and PCT Application PCT/US2003/028100).

For example, the manufacture of a haemostatic bandage (U.S. Pat. No. 6,762,336) involves a multi-step manufacturing process that places fibrinogen and thrombin into separate layers. The purpose of the separate layers was to minimize the fibrinogen/thrombin interaction so fibrin would not be formed during the manufacturing process. The resulting product, although effective, is subject to delamination during shipping and handling. Indeed, this deficiency led to the imposition of an even more complex structure and attendant manufacturing process involving an interrupted layer of thrombin (US Patent Application 20060155234: Haemostatic dressing. MacPhee et al, Jul. 13, 2006). If one could mix fibrinogen and thrombin together in a single step, under conditions that minimize fibrin formation, then a simpler manufacturing process that would produce a more robust product, at a reduced manufacturing cost and complexity, with an increased throughput would be possible.

However, as explained above, fibrin, the usual product of the mixing of fibrinogen and thrombin, is itself only weakly haemostatic (D. B. Kendrick, Blood Program in WW II Washington. D.C.: Office of the Surgeon General, Department of Army; 1989. 363-368 & Tissue Sealants Available Today. MacPhee, M & Wilmer, K. in Tissue Glues In Cosmetic Surgery. Renato Saltz & Dean M. Toriumi, Eds. Quality Medical Publishing, Inc. 2004) as compared to the effectiveness of a mixture of fibrinogen, thrombin and factor XIII that does not polymerize before contact with the wound to be treated but rather polymerizes in situ after placed in contact with the wound. This is the reason that fibrin sealant products are manufactured so as to maintain effective separation between at least the thrombin component and the fibrinogen/factor XIII component(s). This is generally accomplished by either drying and packaging the components separately as with conventional fibrin sealants, or by constructing a structure in which the components are layered upon each other under conditions that prevent their interaction (See U.S. Pat. No. 6,762,336).

The extent to which thrombin has interacted with fibrinogen and factor XIII can be determined by measuring the extent to which the native fibrinogen has undergone conversion to fibrin. One of the direct effects of thrombin upon fibrinogen is to remove several small portions of two of the three protein chains comprising the intact fibrinogen molecule. The result is the release of the peptides referred to as fibrinopeptides a and b. This loss can be determined by several methods known in the art, including the change in the molecular weight of the A-a and B-b chains as they are converted into A & B by the release of the a and b fibrinopeptides respectively. Furthermore thrombin acts upon Factor XIII by removing from it a small peptide, converting the inactive Factor XIII into the active form, known as Factor XIIIa. The effect of Factor XIIIa upon fibrinogen is to form covalent bonds between adjacent fibrinogen γ chains. This converts single γ chain monomers into γ-γ dimers. The resulting loss of the γ monomer and appearance of γ-γ dimers can also be measured by several techniques known to those skilled in the art, with a simple example being the use of electrophoresis to measure the apparent molecular weights of the components of fibrinogen-based compositions.

Thus the extent to which the three components, fibrinogen, thrombin and factor XIII have interacted can be quantified by several methods. Generally, these involve measuring the proportion of conversion of the fibrinogen chains from their native form to their state within fibrin. This can be accomplished by first measuring the amount of native and/or fibrin form in a composition, then repeating the same measurement(s) on the same composition after first placing the composition for a suitable time into an environment in which the reaction of the components will be completed. Dividing the amount of material in the fibrin form in the initial composition by the amount formed by the complete reaction of the composition determines the proportion of the initial composition that had reacted to form fibrin and thus will not contribute significantly to the haemostatic action of the composition. This can be accomplished for example, by measuring the amount of A-a that converts to A, the amount of B-b that is converted into B or the amount of γ-γ dimer formation.

This requirement to prevent the interaction between fibrinogen, thrombin and factor XIII has limited the nature, structures and manufacturing process of fibrinogen-thrombin based products. Furthermore it has led to complex structures and production process Thus new or improved products could be made if it were possible to manufacture a product by mixing fibrinogen±Factor XIII and thrombin together in a manner that limits fibrin formation.

This patent describes monolithic compositions of fibrinogen±factor XIII and thrombin that remain active and capable of reacting with each other to subsequently form fibrin. These compositions are described in liquid, frozen and solid states. Additionally, manufacturing processes by which these components are combined under conditions that minimize fibrin formation. The resulting compositions and their uses are also described.

### III. SUMMARY OF THE INVENTION

It is therefore an object of the present invention to produce compositions comprising fibrinogen±Factor XIII, thrombin and fibrin in suitable relative proportions and absolute quantities that may be used to make an effective wound dressing, such as a monolithic dressing or bandage. It is also an object of the invention to treat patients in need thereof using compositions comprising fibrinogen±Factor XIII, thrombin and fibrin in suitable relative proportions and absolute quantities. Other objects, features and advantages of the present invention will be set forth in the detailed description of preferred embodiments and appended claims that follow, and in part will be apparent from that description or may be learned by the practice of the invention. These objects and advantage of the invention will be attained by the compositions, processes and methods particularly pointed out in the written description and claims hereof.

### IV. DETAILED DESCRIPTION OF THE PREFERRED EMBODIMENTS

### A. Definitions

Unless defined otherwise herein, all technical and scientific terms used herein have the same meaning as is commonly understood by one of skill in the art to which this invention belongs. All patents and publications mentioned herein are incorporated by reference.

“Dressing” as used herein refers to a material applied to a wound with the intension of treating the wound in such a manner as to limit, eliminate or prevent one or more undesirable processes from occurring in or around the application site. This term encompasses related terms such as “bandage”, etc.

“Thrombin” as used herein refers to Coagulation Factor IIa, its pre-cursers and derivatives. Thrombin may be used to convert fibrinogen to Fibrin I. It may also be used to convert Blood Coagulation Factor XIII to Factor XIIIa, which in turn is able to convert Fibrin I to insoluble, cross linked Fibrin II. If a composition contains all three of Fibrinogen, Thrombin and Factor XIII, then the action of Thrombin may be in effect to convert Fibrinogen to Fibrin. As used herein, unless explicitly stated otherwise, the use of “Thrombin” in a process or composition of the invention also encompasses the use of any other substance that is known to those skilled in the art to cause the conversion of Fibrinogen to one or more forms of Fibrin. Illustrative examples of “Thrombin-equivalents” include, but are not limited to, Thrombin-like enzymes found in snake venoms, such as ancrod, batroxobin, calobin and flavoxobin. The selection of Thrombin and/or a Thrombin-equivalent for use in a particular process or composition of the invention may vary and the particular choice required may be made empirically by one skilled in the art.

“Mold” as used herein refers to a structure or container that either restrains the movement of a composition, or defines its extent in one or more dimensions. A mold may be used merely to form a composition into a desired shape. Alternately, a mold may serve both that function and also one or more additional functions, such as providing a component of a system designed to isolate the composition from the surrounding environment, or to protect it from external alteration by heat or physical shock. Accordingly, a mold may be used temporarily for only a portion of the process required to form a composition, which may then be removed from the mold and the mold discarded or re-used. Alternately, a mold may serve the initial function of giving form to the composition, and be employed subsequently as a container or a portion of a container for the product. The molds may have various connectors and ports that allow the introduction of various compositions into the mold, and/or the escape of the interior atmosphere during filling and/or lyophilization or other drying step. The molds may be fabricated into a single piece, or have one or more movable or removable components to facilitate manufacture or storage.

“Filling” as used herein refers to adding one or more components of a composition to a container or mold. Unless otherwise specified, two or more of the components may be mixed prior to addition to the container or mold. Alternately, two or more of the components may be added sequentially or simultaneously to the container or mold. The resulting mixture may be homogenous or incompletely mixed according to the desired function. The volume used to fill a container may be any useful quantity relative to the volume of the container or mold. When the container or mold has at least one dimension that is longer than another, the filling may be performed with the container or mold in any suitable orientation. For example, if the long axis of the container is oriented horizontally, then the filling of said container while in this orientation is said to be “Horizontal”. Conversely, when the filling takes place while the long axis is oriented vertically the filling is said to be ‘Vertical”. The filling can be carried out with a substantial opening to the surrounding atmosphere exists in the container or mold, such that the area of the opening(s) is/are substantially greater than the area of the opening(s) used to fill the container. This is referred to as “Open Filling” or “Open Mold Filling”. In contrast, when there is no opening of the container that connects unimpeded to the surrounding atmosphere the filling of the mold is said to be a “Closed Filling” or “Closed Mold” filling. When the composition(s) to be filled into said closed filling system is introduced under pressure this is referred to as “Injection Mold Filling” or “Injection Molding”. The container or molds may be at or above ambient temperatures during filling, or below ambient temperature so as to facilitate a rapid freezing of the filled components. Filling may be carried out at such a rate as to permit the effective mixing of the components prior to their freezing into a monolithic mass.

“Haemostatic agent” as used herein is a composition or product that when applied to a patient with at least one site of active bleeding results in a reduction in the rate of blood loss.

“Patient” as used herein refers to human or animal individuals in need of medical care and/or treatment.

“Wound” as used herein refers to any damage to any tissue of a patient that results in the loss of brood from the circulatory system. The tissue may be an internal tissue, such as an organ or blood vessel, or an external tissue, such as the skin. The loss of blood may be internal, such as from a ruptured organ, or external, such as from a laceration. A wound may be in a soft tissue, such as an organ, or in hard tissue, such as bone. The damage may have been caused by any agent or source, including traumatic injury, infection or surgical intervention.

“Resorbable material” as used herein refers to a material that is broken down spontaneously and/or by the mammalian body into components which are consumed or eliminated in such a manner as not to interfere significantly with wound healing and/or tissue regeneration, and without causing any significant metabolic disturbance.

“Stability” as used herein refers to the retention of those characteristics of a material that determine activity and/or function.

“Binding agent” as used herein refers to a compound or mixture of compounds that improves the adherence of one layer of an inventive haemostatic dressing to one or more different layers and/or the adherence of the components of a given layer to other components of that layer.

“Solubilizing agent” as used herein refers to a compound or mixture of compounds that improves the dissolution of a protein or proteins in aqueous solvent.

“Filler” as used herein refers to a compound or mixture of compounds that provide bulk and/or porosity to one or more layers of inventive haemostatic dressings.

“Release agent” as used herein refers to a compound or mixture of compounds that facilitates removal of an inventive haemostatic dressing from a manufacturing mold.

“Foaming agent” as used herein refers to a compound or mixture of compounds that produces gas when hydrated under suitable conditions.

“Monolithic” as used herein refers to a composition that is formed so as to have a single layer with all ingredients within that layer. A backing material may be added to the surface of, or within such a composition without changing its designation as ‘monolithic’.

“Dried” refers to a composition that has had enough of the available water removed from it such that the composition is substantially solid, but not frozen. Suitable methods for drying materials are known and/or can be determined by those skilled in the art, and include; evaporation, sublimation, heating, lyophilizing, spinning, electrospinning (see U.S. Pat. No. 1,975,504, J. Electrostatics 35, 151 (1995) and Polymer, 40, (1999)), concentration, spray drying, liquid crystallization, pressing, crystallization and combinations of two or more such techniques.

As used herein, the term “residual moisture” is intended to mean the amount or proportion of “freely-available water” in the composition. Freely-available water present in the composition that is not bound to or complexed with one or more of the non-liquid components of the composition. “Freely-available water” includes intracellular water. The residual moisture contents referenced herein refer to levels determined by the FDA approved, modified Karl Fischer method (Meyer and Boyd, Analytical Chem., 31, 215-219, 1959; May, et al., J. Biol. Standardization, 10, 249-259, 1982; Centers for Biologics Evaluation and Research, FDA, Docket No. 89D-0140, 83-93; 1990).

“Frozen” as used herein refers to a material, at least some part of which that has transitioned from a liquid to a solid state, by the loss of thermodynamic energy. Materials may be partially or completely frozen, depending upon their temperature and time at that temperature. The transition between a liquid to a solid, frozen state may or may not be visually apparent. Furthermore, within material that appears to have been frozen solid, there may remain unfrozen, or “free” water that is imperceptible to the unaided senses. Materials in this state, unless explicitly stated, are still considered to be “frozen”

“Lyophilized” as used herein refers to refers to material that has had some of its available water removed by freezing the material and then reducing the pressure surrounding it. This process is synonymous with “Freeze-drying”. The reduction in the available water may be sufficient that the material may exist as a solid at temperatures at which it would have been a liquid prior to lyophilization.

“Cooling” as used herein refers to the process of lowering the temperature of an object or composition. There are three fundamental processes by which cooling may take place. These are referred to as Convective, Conductive and Radiative cooling (Introduction to the Principals of Heat Transfer, Website available at: http://www.efunda.com/formulae/heat_transfer/home/overview.cfm Jul. 19 2006). In practice it is difficult to cool an object by only one of these mechanisms, however cooling processes can be devised in which one or two of these mechanisms predominate. An example of this is the industrial process of blast cooling or blast freezing. In this process, a large volume of cooled air or other gas is forced past the object(s) to be cooled. The majority of the heat energy removed from the object is transferred to the moving gas and removed via convection. This type of convective cooling is referred to as “Forced Convection”. This form of cooling is often augmented by the introduction into the cooling gas of a cryogenic liquid, such as liquid nitrogen, to produce a very low temperature cooling gas and reduce the cooling or freezing time. Conductive cooling can predominate when a cooled block of material is placed in contact with the object to be cooled. Radiative cooling can dominate when an object to be cooled is placed in close proximity, but not in contact, with a cooled object.

### B. Preferred Embodiments

In accordance with these and other objects, a first embodiment of the present invention is directed to a haemostatic composition comprising a frozen mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis. The particular amount of fibrin that may be contained in the composition may vary depending upon the ultimate intended use of the composition. Suitable “insufficient amounts” of fibrin may therefore be determined empirically by one skilled in the art, depending upon the intended use thereof.

A second embodiment of the present invention is directed to a dried haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin, upon application to or mixing with bodily fluids or an exogenous aqueous fluid, preferably containing an effective amount of Ca⁺², and/or application to injured tissue, to provide effective hemostasis.

A third embodiment of the present invention is directed to a lyophyllized haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

A fourth embodiment of the present invention is directed to a haemostatic monolithic dressing for treating wounded tissue in a patient which comprises an effective mixture of dried fibrinogen and thrombin, with or without Factor XIII, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

A fifth embodiment of the present invention is directed to a haemostatic monolithic dressing for treating wounded tissue in a patient which comprises an effective mixture of lyophyllized fibrinogen and thrombin, with or without Factor XIII, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

A sixth embodiment of the present invention is directed to a haemostatic monolithic dressing for treating wounded tissue in a patient which comprises a backing material, an effective mixture of dried fibrinogen and thrombin, with or without Factor XIII, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

A seventh embodiment of the present invention is directed to a haemostatic monolithic dressing for treating wounded tissue in a patient which comprises a backing material, an effective mixture of lyophillized fibrinogen and thrombin, with or without Factor XIII, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

An eighth embodiment of the present invention is directed to a haemostatic composition for treating wounded tissue in a patient which comprises an effective mixture of fibrinogen and thrombin, with or without Factor XIII, wherein one or more components of the mixture are non-homogenously distributed throughout said mixture, and which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

A ninth embodiment of the present invention is directed to a haemostatic monolithic dressing for treating wounded tissue in a patient which comprises an effective mixture of fibrinogen and thrombin, with or without Factor XIII, wherein one or more components of the mixture are non-homogenously distributed throughout said mixture, and which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

A tenth embodiment of the present invention is directed to a haemostatic monolithic composition for treating wounded tissue in a patient which comprises an effective mixture of fibrinogen and thrombin, with or without Factor XIII, wherein one or more components of the mixture are non-homogenously distributed throughout said mixture according to a continuously varying gradient, and which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

An eleventh embodiment of the present invention is directed to a monolithic dressing for treating wounded tissue in a patient which comprises an effective mixture of fibrinogen and thrombin, with or without Factor XIII, wherein one or more components of the mixture are non-homogenously distributed throughout said mixture according to a continuously varying gradient, and which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

A twelfth embodiment of the present invention is directed to a haemostatic monolithic composition for treating wounded tissue in a patient which comprises an effective mixture of dried fibrinogen and thrombin, with or without Factor XIII, wherein one or more components of the mixture are non-homogenously distributed throughout said mixture, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis

A thirteenth embodiment of the present invention is directed to a haemostatic monolithic dressing for treating wounded tissue in a patient which comprises an effective mixture of dried fibrinogen and thrombin, with or without Factor XIII, wherein one or more components of the mixture are non-homogenously distributed throughout said mixture, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

A fourteenth embodiment of the present invention is directed to a haemostatic composition for treating wounded tissue in a patient which comprises an effective mixture of lyophyllized fibrinogen and thrombin, with or without Factor XIII, wherein one or more components of the mixture are non-homogenously distributed throughout said mixture, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

A fifteenth embodiment of the present invention is directed to a haemostatic monolithic dressing for treating wounded tissue in a patient which comprises an effective mixture of lyophyllized fibrinogen and thrombin, with or without Factor XIII, wherein one or more components of the mixture are non-homogenously distributed throughout said mixture, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

A sixteenth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: filling a suitable mold with the mixture components; and applying sufficient cooling to the mold and or composition so as to freeze the mixture into a monolithic mass before the formation of sufficient fibrin to prohibit its use as a haemostatic agent occurs. One preferred method of doing this is by cooling the two active bulk substances below 0° C. and then combining the two slurries prior to dispensing into the mold and freezing. Particular ways of accomplishing this include mixing together pre-cooled fibrinogen and pre-cooled thrombin (both pre-cooled to 2°-8° C.) in a pre-cooled dispensing vessel held between 0° C. and above the freezing point of the mixed solution (the ice slurry could preferably be mixed to ensure homogeneity) and then snap frozen or the fibrinogen and thrombin could be individually pre-cooled to a temperature below 0° C. and above the freezing point of the solution to form an ice/water slurry and then the two slurries could then be mixed together (again, the ice slurries could be mixed to ensure homogeneity prior to dispensing) and then snap frozen. The parameters to be optimized include;
1. temperature and time that pre-cooled fibrinogen and thrombin are stable;
2. temperature and time Fibrinogen and thrombin ice slurries are stable;
3. temperature and time the mixed fibrinogen and thrombin ice slurries are stable; and
4. chemical additive(s) which can lower the freezing temperature of each mixture or the combined mixture.

While not wishing to be bound by any theory of operability, according to Seegers et al (Arch Biochem Biophys 128:194-201, 1968), the optimal pH for Thrombin activity is near or at pH 8.0 for turnover of chromogenic substrates, with activity found across the pH range>5 and <11 (activity reached zero at the extremes of this range). Most chromogenic assays of Thrombin activity are buffered at pH 8.3 to be at or near this optimum condition. Similar pH-dependence for clotting of fibrinogen is reported by Mihalyi et al (Biochemistry 30:4753-4762, 1991). Maximum rate was near pH 7.8, and the rate was only slightly slower at pH 8.8. While clotting assays are usually conducted at pH 7.4, this is probably done to mimic physiological conditions in the blood stream, not related to the higher pH for maximum thrombin clotting activity.

A seventeenth embodiment of the present invention is directed to a frozen haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said composition comprising the mixture of components having a pH in the range of 1-6 (i.e. ≧1 and ≦6) or having a pH>10. Particularly preferred examples of this and similar embodiments may have a pH in the range of 1-5 or a pH>11.

An eighteenth embodiment of the present invention is directed to a dried haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon exposure to an aqueous environment to provide effective hemostasis, said composition comprising the mixture of components having a pH>1 and <6 or a pH>10. As noted above, particularly preferred examples include a pH in the range of 1 to 5 or a pH>11.

A nineteenth embodiment of the present invention is directed to a lyophyllized haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon exposure to an aqueous environment to provide effective hemostasis, said composition comprising the mixture of components having a pH>1 and <6 or a pH>10. As noted above, particularly preferred examples include a pH in the range of 1-5 or a pH>11.

A twentieth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: formulating the mixture components such that when mixed, they form a mixture having a pH>1 and <6 or a pH>10; filling a suitable mold with the mixture components; and applying sufficient cooling to the mold and/or to the mixture components so as to freeze the mixture into a monolithic mass before excess fibrin formation occurs.

A twenty-first embodiment of the present invention is directed to a method for producing a haemostatic composition comprising a dried mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: formulating the mixture components such that when mixed, they form a mixture having a pH>1 and <6 or a pH>10; filling a suitable mold with the mixture components; and applying sufficient cooling to the mold and/or to the mixture components so as to freeze the mixture into a monolithic mass before excess fibrin formation occurs, and subsequently drying the mixture

A twenty-second embodiment of the present invention is directed to a method for producing a lyophilized haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: formulating the mixture components such that when mixed, they form a mixture having a pH>1 and <6 or a pH>10; filling a suitable mold with the mixture components; and applying sufficient cooling to the mold and/or to the mixture components so as to freeze the mixture into a monolithic mass before excess fibrin formation occurs, and subsequently lyophilizing the mixture to a suitably low residual moisture level.

In certain embodiments of the present invention, the concentration of sodium ion (Na⁺) may be varied. While not wishing to be bound by any theory of operability, sodium ion is known to bind to Thrombin and cause an allosteric shift from a “slow” form of Thrombin (predominating at zero or very low sodium ion concentrations) to a “fast” form at 0.2M sodium ion content. The slow form does not clot fibrinogen quickly, but activates Protein C well and thereby inhibits the coagulation process (i.e. is anticoagulant). The fast form clots fibrinogen quickly (i.e. is procoagulant), but activates Protein C poorly, and does not foster the anticoagulation system of plasma.

While not wishing to be bound by any theory of operability, in certain embodiments of the invention, the sodium content of the Thrombin and/or fibrinogen components (alone or in combination with other process variables) can be manipulated to foster or inhibit clotting. For example, low sodium content can inhibit clotting of fibrinogen as the components are mixed to form a monolithic bandage. Later, the natural concentration of components that occurs when the mixture is subjected to lyophilization can increase the sodium content to foster clotting of fibrinogen when the dressing is hydrated by application to wounded tissue.

In early work by Di Cera and colleagues, the authors used 0.2M NaCl to put thrombin into the fast form, whereas they used 0.2M choline chloride (no Na⁺) to study the slow form. An example can be seen in Dang Q D, Vindigni A and Di Cera E, An allosteric switch controls the procoagulant and anticoagulant activities of thrombin, Proc Natl Acad Sci USA, 92:5977-5981, 1995.

A region of Na⁺concentration where this allosteric shift takes place is at the Na⁺concentration of normal plasma. (For a discussion see Di Cera E, Thrombin Interactions, Chest 124 Supplement:11S-17S, 2003). At the Na⁺content in normal blood (140 mEq/L), the slow and fast forms are present at a 2:3 ratio (40% slow, 60% fast). Hypernatremia (Na⁺>145 mEq/L) is often associated with increased fibrinogen cleavage and venous thrombosis. Hyponatremia (Na⁺<135 mEq/L) has reportedly been associated with increased bleeding in infants (Di Cera², pp 14S-15S).

A twenty-third embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: formulating the mixture components such that when mixed, they form a mixture with a low sodium content; filling a suitable mold with the mixture components; and applying sufficient cooling to the mold and/or to the mixture components so as to freeze the mixture into a monolithic mass before excess fibrin formation occurs.

A twenty-fourth embodiment of the present invention is directed to a haemostatic composition comprising; a frozen mixture of fibrinogen and thrombin, with or without Factor XIII, with a low sodium content which contains insufficient fibrin to prohibit its effective use as a haemostatic agent, and which further retains the ability to convert sufficient fibrinogen to fibrin upon thawing to provide effective hemostasis, said composition comprising a mixture with a low sodium content.

A twenty-fifth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: formulating the mixture components such that when mixed, they form a mixture comprising substantially no Ca⁺² or Mg⁺²; filling a suitable mold with the mixture components; and applying sufficient cooling to the mold and/or to the mixture components so as to freeze the mixture into a monolithic mass before excess fibrin formation occurs.

A twenty-sixth embodiment of the present invention is directed to a haemostatic composition comprising a frozen mixture of fibrinogen and thrombin, with or without Factor XIII, said mixture further comprising substantially no Ca⁺² or Mg⁺²; which contains insufficient fibrin to prohibit its effective use as a haemostatic agent, and which further retains the ability to convert sufficient fibrinogen to fibrin upon thawing to provide effective hemostasis.

A twenty-seventh embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, said filling being conducted in the vertical direction; and applying sufficient cooling to the mold and/or to the mixture components so as to freeze the mixture into a monolithic mass before excess fibrin formation occurs.

A twenty-eighth embodiment of the present invention is directed to a method for producing a haemostatic composition comprising a mixture of fibrinogen and thrombin, with or without Factor XIII, which contains insufficient fibrin to prohibit its effective use as a agent, and which further retains the ability to convert sufficient fibrinogen to fibrin upon thawing to provide effective hemostasis, said method comprising the steps of: suitably formulating the mixture components; filling a suitable mold with the mixture components, said filling being conducted in the horizontal direction; and applying sufficient cooling to the mold and/or to the mixture components so as to freeze the mixture into a monolithic mass before excess fibrin formation occurs.

A twenty-ninth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components; and applying sufficient convective cooling to the mold and/or to the mixture components so as to freeze the mixture into a monolithic mass before excess fibrin formation occurs.

A thirtieth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components; and applying sufficient conductive cooling to the mold and/or to the mixture components so as to freeze the mixture into a monolithic mass before excess fibrin formation occurs.

A thirty-first embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components; and applying sufficient radiative cooling to the mold and/or to the mixture components so as to freeze the mixture into a monolithic mass before excess fibrin formation occurs.

A thirty-second embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components; and applying sufficient blast cooling to the mold and/or to the mixture components so as to freeze the mixture into a monolithic mass before excess fibrin formation occurs.

A thirty-third embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, and applying sufficient cooling to one or more sides of the mold and/or to the mixture components so as to freeze the mixture into a monolithic mass before excess fibrin formation occurs.

A thirty-fourth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, and applying sufficient cooling to two or more opposing sides of the mold and/or to the mixture components so as to freeze the mixture into a monolithic mass before excess fibrin formation occurs.

A thirty-fifth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, and applying sufficient cooling to one or more sides of the mold and/or to the mixture components so as to freeze the mixture into a monolithic mass before excess fibrin formation occurs, said freezing occurring in the direction parallel to the shortest axis of the mold.

A thirty-sixth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, and applying sufficient cooling to one or more sides of the mold and/or to the mixture components so as to freeze the mixture into a monolithic mass before excess fibrin formation occurs, said freezing occurring in the direction parallel to the longest axis of the mold.

A thirty-seventh embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, and applying sufficient cooling to one or more sides of the mold and/or to the mixture components so as to freeze the mixture into a monolithic mass before excess fibrin formation occurs, said freezing occurring in the direction parallel to the second shortest axis of the mold.

A thirty-eighth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, and applying sufficient cooling to one or more sides of the mold and/or to the mixture components so as to freeze the mixture into a monolithic mass before excess fibrin formation occurs, said freezing occurring in the directions parallel to two or more of the axes of the mold.

A thirty-ninth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, and applying sufficient cooling to one or more sides of the mold and/or to the mixture components so as to freeze the mixture into a monolithic mass before excess fibrin formation occurs, said freezing occurring in the directions parallel to all of the axes of the mold.

A fortieth embodiment of the present invention is directed to a haemostatic monolithic dressing for treating wounded tissue in a patient which comprises an effective mixture of frozen fibrinogen and thrombin, with or without Factor XIII, which contains insufficient fibrin to prohibit its effective use as a haemostatic agent, and which further retains the ability to convert sufficient fibrinogen to fibrin, upon application to and/or mixing with a bodily fluid or an exogenous aqueous fluid and/or upon application to injured tissue, to provide effective hemostasis.

A forty-first embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components; and applying sufficient blast cooling to the mold and/or to the mixture components so as to freeze the mixture into a monolithic mass before excess fibrin formation occurs.

A forty-second embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components; and applying sufficient convective cooling with a cryogenic gas to the mold and/or to the mixture components so as to freeze the mixture into a monolithic mass before excess fibrin formation occurs.

A forty-third embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; producing dried filaments of the components via centrifugal spinning; and combining filaments of the components into a single structure capable of producing effective hemostasis by any of the means known and available to those of skill in the art.

A forty-fourth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably co-formulating the mixture components; producing dried filaments of the co-formulated components via centrifugal spinning; and combining filaments of the components into a single structure capable of producing effective hemostasis by any of the means known and available to those of skill in the art.

A forty-fifth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; producing dried filaments of the components via electrospinning; and combining filaments of the components into a single structure capable of producing effective hemostasis by any of the means known and available to those of skill in the art.

A forty-sixth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably co-formulating the mixture components; producing dried filaments of the co-formulated components via electrospinning; and combining filaments of the components into a single structure capable of producing effective hemostasis by any of the means known and available to those of skill in the art.

A forty-seventh embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; freezing said compositions, producing small fragments of the components; and combining said fragments of the components into a single structure capable of producing effective hemostasis by any of the means known and available to those of skill in the art, including, but not limited to, pressing the fragments into a single cohesive mass, with or without the addition of sufficient.exogenous heat to facilitate partial melting of the fragments, followed by sufficient cooling to freeze the partially melted fragments into monolithic mass before excess fibrin formation occurs, and subsequently lyophilizing the mixture to a suitably low residual moisture level.

A forty-eighth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably co-formulating the mixture components into a single mass; freezing said mass, producing small fragments of said mass; and combining said fragments into a single structure capable of producing effective hemostasis by any of the means known and available to those of skill in the art, including, but not limited to, pressing the fragments into a single cohesive mass, with or without the addition of sufficient.exogenous heat to facilitate partial melting of the fragments, followed by sufficient cooling to freeze the partially melted fragments into monolithic mass before excess fibrin formation occurs, and subsequently lyophilizing the mixture to a suitably low residual moisture level.

A forty-ninth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; freezing said mixture components in such a manner as to simultaneously produce small fragments of the components by any of the means known to those of skill in the art, including, but not limited to, spraying the mixture in the presence of an expanding cryogenic gas, such as liquid nitrogen or compressed carbon dioxide, and combining said fragments of the components into a single structure capable of producing effective hemostasis by any of the means known and available to those of skill in the art, including, but not limited to, pressing the fragments into a single cohesive mass, with or without the addition of sufficient.exogenous heat to facilitate partial melting of the fragments, followed by sufficient cooling to freeze the partially melted fragments into monolithic mass before excess fibrin formation occurs, and subsequently lyophilizing the mixture to a suitably low residual moisture level.

A fiftieth embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably co-formulating the mixture components into a single mass; freezing said mass in a manner as to simultaneously produce small fragments of said mass by any of the means known to those of skill in the art, including, but not limited to, spraying the mixtures in the presence of an expanding cryogenic gas, such as liquid nitrogen or compressed carbon dioxide, and combining said fragments into a single structure capable of producing effective hemostasis by any of the means known and available to those of skill in the art, including, but not limited to, pressing the fragments into a single cohesive mass, with or without the addition of sufficient.exogenous heat to facilitate partial melting of the fragments, followed by sufficient cooling to freeze the partially melted fragments into monolithic mass before excess fibrin formation occurs, and subsequently lyophilizing the mixture to a suitably low residual moisture level.

Additionally, while not wishing to bound by any theory of operability, in certain embodiments of the present invention, volatile buffers can be utilized to maintain the pH of a protein solution, and can be removed from the protein when the solution is dried by lyophilization or other evaporative process. For example, a protein can be buffered at pH 5 with ammonium acetate, and upon drying the ammonium acetate evaporates and the pH reverts to that of nonvolatile buffering components (e.g., the protein itself or other constituent buffers).

Similarly, one ore more of the protein components of the composition may be suspended in a volatile non-aqueous solvent, thereby partitioning it from the other components during mixing to form a monolithic composition. If this organic solvent is volatile, then it can be removed by drying, leaving all the components in a substantially organic composition that is capable or reacting to form fibrin for an effective haemostatic effect upon re-hydration.

A fifty-first embodiment of the present invention is directed to a method for producing a lyophilized haemostatic composition comprising a mixture of fibrinogen and thrombin, with or without Factor XIII, which contains insufficient fibrin to prohibit its effective use as a haemostatic agent, said composition comprising the mixture of components having a pH<6 or a pH>8 and a volatile buffer having a pH<6 (e.g. ammonium acetate) or a pH>8 (e.g. ammonium bicarbonate) which is removed by the lyophilization process, leaving a solid at neutral pH which retains the ability to convert sufficient fibrinogen to fibrin upon reconstitution to provide effective hemostasis.

The Lyotropic Series of chemicals is a ranking of chemical compounds or ions based on their effect on the structure and aggregation state of macromolecules.

At one end of the Lyotropic Series are chaotropic agents (also called “salting-in”, “structure-breaking” or “destabilizing” chemicals) that reduce the interactions between proteins or protein domains, and therefore reduce the tendency of proteins to interact or aggregate. Examples of chaotropic agents include, but are not limited to: urea, guanidine, arginine, thiocyanate, bisulfite, iodide, nitrate, calcium, magnesium, and chloride ions.

At the other end of the Lyotropic Series are “anti-chaotropes” (usually called “salting-out”, “structure making” or “stabilizing” chemicals) which tend to enhance the interaction, aggregation and/or precipitation of proteins. Anti-chaotropic anions include, but are not limited to: sulfate (e.g. ammonium sulfate), phosphate, citrate, and EDTA. Cations include quaternary amines, ammonium and to a lesser extent sodium and potassium ions.

The Lyotropic Series was first described by Von Hippel and Schleich (“The effects of neutral salts on the structure and conformational stability of macromolecules in solution”, in Structure and Stability of Biological Macromolecules, Timasheff and Fasman (eds), Vol 2, Marcel Dekker, New York, p 417-574). One clear protein application was summarized by Busby et al (J Biol Chem 256:12140-12147, 1981). U.S. Pat. No. 6,447,774 (Metzner et al) claims the use of chaotropes to stabilize liquid formulations of fibrinogen and Factor XIII, as part of a storage stable liquid fibrin sealant.

While not wishing to be bound by any theory of operability, in certain embodiments of the present invention, chaotropes may help to prevent the formation of fibrin during mixing of fibrinogen and thrombin proteins to prepare the haemostatic composition. While not wishing to be bound by any theory of operability, in certain other embodiments, anti-chaotropes may enhance these protein-protein interactions. The composition of the protein mixture can therefore be adjusted to achieve a beneficial balance between chaotropic and anti-chaotropic compounds to achieve the desired properties of the protein mixture. It is to be understood that the presence of said components must not have unacceptable deleterious affects on the fibrinogen, Factor XIII or thrombin under the selected conditions.

A fifty-second embodiment of the present invention is directed to a frozen (or dried or lyophilized) haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing (or upon application to or mixing with a bodily fluid or an exogenous aqueous fluid and/or upon application to injured tissue) to provide effective hemostasis, wherein one or more of the components is a chaotropic compound.

A fifty-third embodiment of the present invention is directed to a method for producing a lyophilized haemostatic composition comprising a mixture of fibrinogen and thrombin, with or without Factor XIII, which contains insufficient fibrin to prohibit its effective use as a haemostatic agent, said composition comprising the mixture of components containing one or more chaotropic compounds together with one or more ant-chaotropic compounds, which retains the ability to convert sufficient fibrinogen to fibrin upon reconstitution to provide effective hemostasis.

Additionally, in each of the embodiments of the present invention, in addition to the active components of the mixture, the compositions and mixtures may also optionally contain one or more suitable foaming agents, such as a mixture of citric acid and sodium bicarbonate. Additional agents that generate gas when thawed and/or hydrated are known to those skilled in the art.

Each of the inventive haemostatic dressings may also further comprise a backing material on the side of the bandage opposite the wound-facing side. The backing material may be affixed with a physiologically-acceptable adhesive, or may be self-adhering (e.g., by having a sufficient surface static charge). The backing material may be a resorbable or non-resorbable material. Preferably the backing is resorbable, such as collagen, fibrin, fibrinogen, Vicryl™ or Dexon™. The backing material may be proteinacious, such as keratin, silk etc.

Any suitable resorbable material known to those skilled in the art may be employed in the present invention. For example, the resorbable material may be a proteinaceous substance, such as silk, fibrin, keratin, collagen and/or gelatin, or a carbohydrate substances, such as alginates, chitin, cellulose, proteoglycans (e.g. poly-N-acetyl glucosamine), glycolic acid polymers, lactic acid polymers, or glycolic acid/lactic acid co-polymers. Specific resorbable material(s) for a particular application may be selected empirically by those skilled in the art.

Preferably, the resorbable material is a carbohydrate substance. Illustrative examples of particularly preferred resorbable materials are sold under the tradenames Vicryl™ (Poly(Lactide-Co-Glycoside), a glycolic acid/lactic acid copolymer) and Dexon™. (glycolic acid polymer).

The backing material may be in the form of a solid sheet or composed of individual strands or fibers formed into a cloth or felt-like material, woven, knitted, extruded, spun; electrospun, combed, compressed or felted. Suitable pore sizes of the resulting material may be determined empirically by one of ordinary skill in the art and may range in diameter from 2000 microns to less than one nanometer. More preferably, they may range from 1000 to one microns, and more preferable from 200 to 700 microns, and most preferably from 230 to 635 microns.

In certain embodiments of the present invention the backing material may be within the mass of the haemostatic mixture. Preferably the backing material is located substantially on the side opposite the tissue-contacting face. In another preferred embodiment the backing material is located substantially within the center of the haemostatic mass.

The proteinacious components of the inventive compositions may originate in any animal species, and may be natural, modified, derivatized, recombinant, or transgenic in nature. Preferably the species of origin of naturally-derived materials is human. If the material is recombinant or transgenic in nature, preferably the species of origin of the primary amino acid sequence is human. Additional preferred species include bovine and porcine.

The fibrinogen employed in the inventive haemostatic compositions is preferably human, but any suitable preparation may be utilized. Such suitable preparations may include derivatives and metabolites, such as Fibrin I. A specific fibrinogen or fibrinogen containing composition for a particular application may be selected empirically by one of ordinary skill in the art. The fibrinogen may also contain Factor XIII at a level sufficient to produce adequate cross linking of the fibrin strands to each other, and to the tissue to which the composition is applied.

The thrombin employed in the inventive haemostatic compositions is preferably human, but any suitable preparation may be utilized. A specific thrombin or thrombin containing composition for a particular application may be selected empirically by one of ordinary skill in the art. Additionally, in each of the embodiments of the present invention, thrombin may be replaced by any of the thrombin-equivalents known by those skilled in the art to be activators of fibrinogen conversion to fibrin. Illustrative examples of such agents are snake venoms, such as batroxiben. A specific activator of fibrin formation for a particular application may be selected empirically by one skilled in the art.

In each of the embodiments of the present invention, one or more of the protein components of the composition or mixture can be coated with a slowly dissolving coat of an acceptable inactive excipient. By tailoring the composition and the thickness of the coating, the duration of partitioning of the coated component can be adjusted to coincide with the manufacturing process such that there is insufficient fibrin formation to significantly reduce the haemostatic effectiveness of the composition upon re-hydration.

Each of the inventive haemostatic bandages may also further comprise a backing material on the side of the bandage opposite the wound-facing side. The backing material may be affixed with a physiologically-acceptable adhesive or may be self-adhering (e.g by having a sufficient surface static charge). The backing material may be a resorbable material or a non-resorbable material, such as a silicone patch or plastic. Preferably, the backing material is a resorbable material.

Additionally, in each of the embodiments of the present invention, in addition to the active components of the mixture, one or more inactive carrier or filler materials may also be incorporated into the formulation. Preferred examples include albumin, Immunoglobulin, sucrose, manitol, xylose, xylol, Chitosan and its derivatives, collagen and its derivatives, polysorbate, alginates and Fibronectin.

Additionally, in each of the embodiments of the present invention, in addition to the active components of the mixture, one or more binding materials may also be incorporated into the formulation. Preferred examples include albumin, sucrose, Chitosan and its derivatives, collagen and its derivatives, polysorbate and Fibronectin.

Additionally, in each of the embodiments of the present invention, in addition to the active components of the mixture, the composition may also optionally further comprise a release agent which may optimally be applied to the mold prior to filling with the proteinacious materials. A preferred release agent is sucrose. Others include, but are not limited to; chitosan and its derivatives, dextrose, silocone-containing compounds, detergents and oils.

Additionally, in each of the embodiments of the present invention, in addition to the active components of the mixture, one or more solubilizing materials may also be incorporated into the formulation. Preferred examples include albumin, sucrose, Chitosan and its derivatives, detergents, tensides, PEG, PPG and polysorbate.

For all of the components of the inventive embodiments, suitable materials may be obtained from various sources, and purified by any of the purification methods known to those skilled in the art. An important component of such methods include techniques that avoid, reduce or inactivate pathogens within these materials, including bacteria, molds, spores, viruses and prions.

Alternatively, a physiologically-acceptable adhesive may applied to the resorbable material and/or the backing material (when present) and the fibrinogen layer(s) and/or the thrombin layer(s) subsequently affixed thereto.

In one embodiment of the inventive bandage, the physiologically-acceptable adhesive has a shear strength and/or structure such that the resorbable material and/or backing material can be separated from the fibrinogen layer and/or the thrombin layer after application of the bandage to wounded tissue. In another embodiment, the physiologically-acceptable adhesive has a shear strength such that the resorbable material and/or backing material cannot be separated from the fibrinogen layer and/or the thrombin layer after application of the bandage to wounded tissue.

Suitable fibrinogen and thrombin may be obtained from human or mammalian plasma by any of the purification methods known and available to those skilled in the art; from supernatants or pastes of recombinant tissue culture, viruses, yeast, bacteria, or the like that contain a gene that expresses a human or mammalian plasma protein which has been introduced according to standard recombinant DNA techniques; or from the fluids (e.g. blood, milk, lymph, urine or the like) of transgenic animals that contain a gene that expresses human fibrinogen and/or human thrombin which has been introduced according to standard transgenic techniques.

As a general proposition, the purity of the fibrinogen and/or the thrombin for use in the inventive haemostatic dressing will preferably be an appropriate purity known to one of ordinary skill in the relevant art to lead to the optimal efficacy and stability of the protein. Preferably, the fibrinogen and/or the thrombin has been subjected to multiple chromatographic purification steps, such as affinity chromatography and preferably immunoaffinity chromatography, to remove substances which cause fragmentation, activation and/or degradation of the fibrinogen and/or the thrombin during manufacture, storage and/or use. Illustrative examples of such substances that are preferably removed by purification include protein contaminants, such as inter-alpha trypsin inhibitor and pre-alpha trypsin inhibitor; non-protein contaminants, such as lipids; and mixtures of protein and non-protein contaminants, such as lipoproteins.

The concentration of the fibrinogen and/or the thrombin employed in the inventive haemostatic composition or dressing is also preferably selected to optimize both the efficacy and stability thereof, as may be determined empirically by one skilled in the relevant art. During use of an inventive haemostatic bandage, the fibrinogen and the thrombin are preferably activated at the time the bandage is applied to the wounded tissue by the endogenous fluids of the patient escaping from the hemorrhaging wound. Alternatively, in situations where fluid loss from the wounded tissue is insufficient to provide adequate hydration of the protein layers, the fibrinogen and or the thrombin may be activated by a suitable, physiologically-acceptable liquid, optionally containing any necessary co-factors and/or enzymes, prior to or during application of the haemostatic bandage to the wounded tissue.

In addition, one or more supplements may also be contained in the inventive haemostatic composition, such as growth factors, drugs, polyclonal and monoclonal antibodies and other compounds. Illustrative examples of such supplements include, but are not limited to: antibiotics, such as tetracycline and ciprofloxacin, amoxicillin, and metronidazole; anticoagulants, such as activated protein C, heparin, prostacyclin (PGI.sub.2), prostaglandins, leukotrienes, antithrombin III, ADPase, and plasminogen activator; steroids, such as dexamethasone, inhibitors of prostacyclin, prostaglandins, leukotrienes and/or kinins to inhibit inflammation; cardiovascular drugs, such as calcium channel blockers, vasodilators and vasoconstrictors; chemoattractants; local anesthetics such as bupivacaine; and antiproliferative/antitumor drugs such as 5-fluorouracil (5-FU), taxol and/or taxotere; antivirals, such as gangcyclovir, zidovudine, amantidine, vidarabine, ribaravin, trifluridine, acyclovir, dideoxyuridine and antibodies to viral components or gene products; cytokines, such as .alpha.- or .beta.- or .gamma.-Interferon, .alpha.- or .beta.-tumor necrosis factor, and interleukins; colony stimulating factors; erythropoietin; antifungals, such as diflucan, ketaconizole and nystatin; antiparasitic gents, such as pentamidine; anti-inflammatory agents, such as .alpha.-1-anti-trypsin and .alpha.-1-antichymotrypsin; anesthetics, such as bupivacaine; analgesics; antiseptics; and hormones. Other illustrative supplements include, but are not limited to: vitamins and other nutritional supplements; glycoproteins; fibronectin; peptides and proteins; carbohydrates (both simple and/or complex); proteoglycans; antiangiogenins; antigens; lipids or liposomes; oligonucleotides (sense and/or antisense DNA and/or RNA); and gene therapy reagents.

The following examples are illustrative only and are not intended to limit the scope of the invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

### V. EXAMPLES

### Methods of Rapid Freezing of a Fibrinogen and Thrombin Mixture to Minimize Fibrin Formation.

Rapid freezing of a fibrinogen/thrombin mixture can halt the chemical processes producing the formation of fibrin. The development of a rapid freezing system of a fibrinogen/thrombin mixture involves the following steps:

1. Determine the upper allowable limit of fibrin formation within the product. Gamma-gamma dimer formation, A-α to A conversion, and or B-β to B conversion (measures of fibrin formation) can be observed in the manufacture of the dressing. An upper limit for fibrin formation beyond which dressing performance deteriorates is established to set specifications for the mixing of fibrinogen and thrombin.

2. Develop a dispensing system for the fibrinogen and thrombin that is sufficiently rapid to limit fibrin formation to the level established in step 1 above.

3. Develop a rapidly freezing method which limit fibrin formation to a level within the fibrin specification.

The fibrinogen/thrombin mixture can also be subjected to freeze drying. This allows for room temperature storage of the product. The product can then be activated by the end user with aqueous solutions.

This process can be scaled for various size and shape molds. In this manner, this manufacturing method can be used for different products and applications.

This process can also be used as part of a high throughput system, which will reduce manufacturing costs.

Developing a Fibrin Formation Specification

A specification establishing the upper permissible limit of gamma-gamma dimer formation specification allows for rapid screening of new manufacturing procedures. The upper limit of dimer formation for the fibrin specification is set by manufacturing a fibrin sealant bandage similar to the ones known in the art, but titrating varying amounts of thrombin into the fibrinogen layers of the bandages during the manufacturing process. Overall thrombin concentration in the bandage is kept constant by decreasing the concentration of thrombin in its layer proportionally. Gamma-gamma dimer formation is determined and those particular bandages that pass QA/QC testing are then used to establish the maximum acceptable gamma-gamma dimer levels.

Alternatively, the dressings produced by the new production processes are tested to determine those that achieve suitable performance. Once these new dressings have been identified, the conditions (geometry, time, efficiency of mixing etc.) of fibrinogen and thrombin mixing and freezing (temperature, mold orientation, number of cooling faces, mold material, coolant etc.) that had been used are then altered to produce various levels of gamma-gamma dimer, and these levels compared with their performance in in vitro and ex vivo assays to determine an acceptable upper limit of gamma-gamma dimer, and hence fibrin, formation in the dressing.

A Dispensing System for Fibrinogen and Thrombin.

Suitable controlled mixing/dispensing equipment is commercially available from various vendors. These systems allow for fine reproducible control of amounts of materials and speed of dispensing. Test dressings are manufactured at various pressures, orifice geometries and numbers, and flow rates to determine the optimum processes for filling the molds. Mixing of the fibrinogen and thrombin occurs as the mold is filled. Pre-chilling of the protein solutions and the rapid freezing of the materials, which will occur as or immediately after the molds are filled, limits the interaction of the fibrinogen and thrombin prior to lyophilization. While this pre-mixing may be incomplete, it should be noted that in previous fibrin sealant based haemostatic bandages, performance was not dependent upon the uniformity of fibrinogen and thrombin pre-mixing. Both a layered bandage (with minimal pre-mixing) and a powder bandage (complete premixing) produced fully functional bandages as determined in the ballistic large animal model⁷ and a swine aortotomy models⁸.

Decreasing Fibrin Formation in the Manufacturing Process by Changing Formulations

The formation of fibrin during dispensing may also be reduced by lowering the thrombin concentration in the formulation. Very high levels of thrombin were used in the previously described layered bandage because the formation of fibrin may slow the diffusion of thrombin through its matrix. This was further reinforced when interrupted thrombin layers were also examined at the ARC (WO 2004 1024195). High concentrations of thrombin were also used to insure thrombin diffusion by mass action. Diffusion issues are eliminated when the thrombin is mixed directly with the fibrinogen in the monolithic dressing. Therefore, there is no need to incorporate high concentrations of thrombin to drive diffusion. The effect is to maintain haemostatic efficacy while decreasing fibrin formation during manufacture. Thrombin concentrations are used that are low enough to keep post-manufacture fibrin levels within the fibrin specifications identified in the experiment described above.

Gradient Manufacturing Studies

A relatively high total level of fibrin formation during manufacture may be permissible as long as the concentration of fibrin is low on the surface of the dressing that faces the wound. A fibrinogen/thrombin gradient manufacturing process is employed to produce this structure. The fibrinogen and thrombin gradient is created as the mold is filled by adjusting the flow rates in such a manner that higher thrombin ratios occur on the non wound-facing side of the dressing.

By this means, various gradients can be constructed, including those with fibrinogen alone on the outer faces and fibrinogen/thrombin mixtures within, thrombin gradients that produce decreasing levels of thrombin as the wound-contacting face is approached, and those that have the opposite orientation.

A dressing is also prepared with the thrombin contained within the center of the mass of dressing material. By first dispensing fibrinogen into a horizontal mold, followed by thrombin, and finishing with another bolus of fibrinogen, a monolithic structure with the thrombin largely deposited in the center of the fibrin sealant mass is constructed. Unlike previous layered structures, there are no layers to delaminate as the material has mixed while in the liquid state prior to freezing.

Mold Orientation: Vertical Filling and Injection Molding

The dressing molds may be mounted either horizontally or vertically. The vertical orientation has the advantage of a gravity-based filling process, and two-sided cooling which reduces the amount of fibrin formation prior to freezing. There are analogous filling and freezing processes used in the food industry (ice cream bars), and thus industrial application of this process is relatively conventional.

Horizontally-oriented molds have several advantages. First, they have been used before for dressing manufacture; secondly, they can be used to manufacture the gradient-style dressing described herein, and finally, the filling and freezing of these molds will utilize technology derived from the injection-molding industry, which is designed for high throughput processing.

Development of a Rapid Freezing Method to Stop the Formation of Fibrin

Chilling rate of ⅛ inch thick protein solutions have been observed at approximately −10° C. per second when placed in contact with a steel block maintained at −60° C. The fibrinogen and thrombin are pre-cooled to ˜4° C. in the dispensing system and thus require less than 3 seconds to freeze. If quicker freezing times are desired, liquid nitrogen chilled blocks (−196° C.) are used. Other methods to decrease freezing time are to mount the molds vertically, between two chilled metal blocks. The vertical mounting doubles the surface area contact. A vertically mounted system has other benefits too. In the vertical system, the fibrinogen and thrombin are dispensed into the mold at minimal flow so the heat transfer rate of the system is not over-taxed. The bottom portion of the mold is frozen before the mold is completely filled.

For the vertical system, vertical slots of cGMP steel are cooled by circulating liquid nitrogen or other suitable coolant. Each square foot can accommodate eight vertical slots and therefore a 2′×6′ freezing unit is capable of freezing 96 dressings. This size of the freezing unit is selected to fit into a 3′×8′ aseptic cGMP laminar flow hood thus reducing the possibility of costly lot rejections due to product contamination.

Inhibiting Fibrin Formation During the Manufacturing Process

In another embodiment of this invention, the fibrinogen and thrombin are mixed together in a manner in which the thrombin is inhibited from reacting with the fibrinogen. This is accomplished by using a thrombin inhibitor that loses its activity when the product is used, such as the following method. Thrombin may be temporarily inhibited by manufacturing a spray dried thrombin particle coated with sucrose. The thrombin particle can be suspended in ethanol, and then the suspension is dispensed in the mold with the fibrinogen. The amount of sucrose coating can be adjusted (determined experimentally) so that it dissolves slowly enough to prevent excess fibrin formation in the brief, low temperature manufacturing of the product, but allows the sucrose/thrombin particles to dissolve within seconds when hydrated by the end user.

Making the System Size Scalable by Creating Various Size and Shape Molds.

The stations in the freezing system are designed to accommodate the largest molds (4″×4″). Smaller molds than these fit into ‘inserts’ in the 4″×4″ stations. Steel or aluminum inserts are inserted into the stations to fill the void volume and maintain a surface contact for heat transfer. The dispensing unit to be utilized is programmable and capable of dispensing any volume of material desired. Thus the system is both size and shape scalable.

Developing a High Throughput System.

The overhead dispensing unit fills 96 molds by gravity in less than 30 minutes. A horizontal dispensing (injection molding) system functions at a similar rate or greater. The slowest part of the operation is the time it takes the operator to load and unload the molds from the freezing unit. Even with this limitation, a single operator is able to produce enough dressings in one 8 hour shift to fill an industrial size freeze drier (˜1,000 4″×4″ dressings per lot). Therefore, a single small unit will suffice to manufacture ten times the annual output of the layered dressing manufacturing unit.

Inhibiting Fibrin Formation by Use of a Suitable pH

The pH of the protein solution(s) used to make the Dressing is adjusted so that it is not optimal for fibrin formation. Doing so during manufacture facilitates mixing the fibrinogen, ±factor XIII and thrombin without producing an amount of fibrin that would result in an unacceptable decreased haemostatic effectiveness. For example, thrombin has an effective pH range between 5-11, with activity significantly reduced at the extremes of this range. Thus, using a pH outside or near the extremes of its activity range greatly diminishes its activity. Products are made using a suitable buffering system, such that the pH lies outside of this range while the buffering capacity is limited, preferably significantly lower than that of blood and other bodily fluids and even intravenous resuscitation fluids, then when the Dressing is reconstituted via bodily fluids or suitable exogenous fluid(s), the buffering capacity of the reconstituting fluids will quickly readjust the products' pH to a value that permits adequate thrombin activity to yield affective product performance.

Inhibiting Fibrin Formation by Use of a Suitable Salt Concentration

The sodium concentration is adjusted so that it is not optimal for fibrin formation. In the absence of suitable sodium concentration, thrombin reverts to a form with a greatly reduced capacity for conversion of fibrinogen and Factor XIII to fibrin and Factor XIIIa respectively. When the product is used in the body, the body's own fluids, or a suitable exogenous fluid, reconstitute the bandage and convert the thrombin to a more active form that permits adequate thrombin activity to yield affective product performance.

Inhibiting Fibrin Formation by Use of a Suitable Salt Concentration and pH

As described in above, formulating the Dressing at a suitable pH or salt concentration reduces fibrin formation during manufacture, while permitting the restoration of an effective level of activity upon reconstitution with bodily fluids or a suitable exogenous fluid. A combination of both these strategies is even more effective. Thus when the product is used in the body, the body's own fluids, or a suitable exogenous fluid, will reconstitute the bandage and convert the thrombin to a more active form that permits adequate thrombin activity to yield affective product performance.

Inhibiting Fibrin Formation by an Absence of Ca⁺² and/or Mg⁺²

The presence of Calcium and/or Magnesium ions are required for the activation of prothrombin to thrombin and for the optimal activity of Factor XIIIa. Both these ions are commonly found in most bodily fluids, including blood. Therefore the manufacture of the Dressing using solutions devoid of or with greatly reduced levels of one or both of these ions permits the mixing of the reagents, without excessive fibrin formation. When the product is used in the body, the body's own fluids, or a suitable exogenous fluid, will reconstitute the bandage with a fluid or fluids containing adequate levels of one or both of these ions to convert the thrombin and/or Factor XIII to a more active form that permits adequate activity to yield effective product performance.

This invention describes a method to mix aqueous fibrinogen, ±factor XIII and thrombin together to form a single mass, under conditions that limit the formation of fibrin. The method employs rapid freezing of the components after mixing to limit fibrin formation. The fibrinogen/Factor XIII and thrombin are kept in separate dispensing units as long as possible. The components are mixed at the nozzles of the dispensing units and are dispensed at a slow enough rate into a casting mold at maintained at freezing temperatures in such a manner that the components freeze on contact with the casting mold.

Since thrombin catalyzes the formation of fibrin, it is necessary to adjust the thrombin concentration in the mixture. The thrombin concentration can always be adjusted low enough to limit fibrin formation, but at very low concentrations there may not be enough thrombin to catalyze fibrin formation by the user of the product. Therefore, the thrombin concentration has to be determined experimentally to establish a concentration that limits fibrin formation during the manufacturing process and yet is sufficiently high enough to be useful to the user of the product.

Inhibiting Fibrin Formation by Use of a Suitable pH with a Volatile Buffer

The pH of the protein solution(s) used to make the Dressing is adjusted with volatile buffer salts so that it is not optimal for fibrin formation during preparation of the mixture, but becomes optimal pH for fibrin formation after lyophilization, during which process the volatile buffer(s) is removed by evaporation. Examples of volatile buffers are ammonium acetate used between pH 4 and pH 7 (Doolittle, Biochem J 94: 742, 1965) and ammonium bicarbonate at pH 8-9.
(http://www.molecularinfor.com/MTM/G/G3/G3-1/G3-1-7.html).

Inhibiting Fibrin Formation by Use of Chaotropic Compounds

The protein solution(s) used to make the Dressing includes chaotropic salts that diminish fibrin formation during preparation of the mixture. It is understood that such chaotropic compounds are to be used in conjunction with other conditions (pH, temperature, other formulation components, protein concentration) to achieve an optimal condition.

### Assays for Measuring Performance

The ability of the dressings to seal an injured blood vessel was determined by an ex vivo porcine arteriotomy (EVPA) performance test, which was first described in U.S. Pat. No. 6,762,336. The EVPA performance test evaluates the ability of a dressing to stop fluid flow through a hole in a porcine artery. While the procedure described in U.S. Pat. No. 6,762,336 has been shown to be useful for evaluating haemostatic dressings, it failed to replicate faithfully the requirements for success in vivo. More specifically, the procedure disclosed in U.S. Pat. No. 6,762,336 required testing at 37° C., whereas, in the real world, wounds are typically cooler than that. This decreased temperature can significantly reduce the rate of fibrin formation and its haemostatic efficacy in trauma victims. See, e.g., Acheson et al., J. Trauma 59:865-874 (2005). The test in U.S. Pat. No. 6,762,336 also failed to require a high degree of adherence of the dressing to the injured tissue. A failure mode in which fibrin forms but the dressing fails to attach tightly to the tissue would, therefore, not be detected by this test. Additionally, the pressure utilized in the procedure (200 mHg) may be exceeded during therapy for some trauma patients. The overall result of this is that numerous animal tests, typically involving small animals (such as rats and rabbits), must be conducted to accurately predict dressing performance in large animal, realistic trauma studies and in the clinical environment.

In order to minimize the amount of time and the number of animal studies required to develop the present invention, an improved ex vivo testing procedure was developed. To accomplish this, the basic conditions under which the dressing test was conducted were changed, and the severity of the test parameters was increased to include testing at lower temperatures (i.e. 29-33° C. vs. 37° C., representing the real physiologic challenge at realistic wound temperatures (Acheson et al., J. Trauma 59:865-874 (2005)), higher pressures (i.e. 250 mmHg vs. 200 mmHg), a longer test period (3 minutes vs. 2 minutes) and larger sized arterial injuries (U.S. Pat. No. 6,762,336 used an 18 gauge needle puncture, whereas the revised procedure used puncture holes ranging from 2.8 mm to 4 mm×6 mm).

In addition, a new test was derived to directly measure adherence of the dressing to the injured tissue. Both these tests showed greatly improved stringency and are thus capable of surpassing the previous ex vivo test and replacing many in vivo tests for efficacy

EVPA Performance Testing

Equipment and Supplies:
In-line high pressure transducer(Ashcroft Duralife™ or equivalent)
Peristaltic pump (Pharmacia Biotech™, Model P-1 or equivalent)
Voltmeter (Craftsman™ Professional Model 82324 or equivalent)
Computer equipped with software for recording pressure or voltage information
Tygon™ tubing (assorted sizes) with attachments
Water bath (Baxter Durabath™ or equivalent), preset to 37° C.
Incubation chamber (VWR™, Model 1400 G or equivalent), preset to 37° C.
Thermometer to monitor temperatures of both water bath and oven
Assorted forceps, hemostats, and scissors
10 cc. and 20 cc. syringes with an approximately 0.6 cm hole drilled in center and smaller hole drilled through both syringe and plunger. This hole, drilled into the end of the syringe, will be used to keep the plunger drawn back and stationary.
O-rings (size 10 and 13)
Plastic Shields to fit the 10 cc and 20 cc syringes (approximately 3.5 cm in length)
P-1000 Pipetman™ with tips
Sphygmomanometer with neonatal size cuff and bladder
Programmable Logic Controller (PLC) to control the pumps to maintain the desired pressure profile (Optional. Manual control may be used if desired.)

Materials and Chemicals
Porcine descending aortas (Pel-Freez Biologicals™, Catalog #59402-2 or equivalent)
Cyanoacrylate glue (Vetbond™, 3M or equivalent)
18-gauge needle(s)
0.9% Saline, maintained at 37° C.
Red food coloring
Vascular Punch(es), 2.8 mm or other
Plastic Wrap

Artery Cleaning and Storage
1. Store arteries at −20° C. until used.
2. Thaw arteries at 37° C. in H₂O bath.
3. Clean fat and connective tissue from exterior surface of artery.
4. Cut the arteries into ˜5 cm segments.
5. The arteries may be refrozen to −20° C. and stored until use.

Artery Preparation for Assay
1. Turn the artery inside-out so that the smooth, interior wall is facing outwards.
2. Stretch a size 13 O-ring over a 20 cc syringe or a size 10 O-ring over a 10 cc syringe with an approximately 0.6 cm (0.25 in) hole drilled into one side.
3. Pull the artery onto the syringe, taking care not to tear the artery or have a too loose fit. The artery should fit snugly to the syringe. Slide another O-ring of the same size onto the bottom of the syringe
4. Carefully pull both O-rings over the ends of the artery. The distance between the O-rings should be at least 3.5 cm
5. Using the blade of some surgical scissors, gently scrape the surface of the artery in order to roughen the surface of the artery.
6. Use a 18-gauge needle to poke a hole through the artery over the site of the hole in the syringe barrel (see note above)
7. The tip of the biopsy punch is inserted through the hole in the artery. Depress the punch's plunger to make an open hole in the artery. Repeat a couple of times to ensure that the hole is open and free of connective tissue.
8. Patch holes left by collateral arteries. Generally this is done by cutting a patch from a latex glove and gluing it over the hole with cyanoacrylate glue. Allow the glue to cure for at least 10 minutes.
9. Place the artery in the warmed, moistened container and place in the incubation chamber. Allow the arteries to warm for at least 30 minutes.

Solution and Equipment Preparation
1. Check to see that the water bath and incubation chamber are maintained at 29-33° C.
2. Make sure that there is sufficient 0.9% saline in the pump's reservoir for completion of the day's assays. Add more if needed.
3. Place 0.9% saline and 0.9% saline with a few drops of red food coloring added into containers in a water bath so that the solutions will be warmed prior to performing the assay.
4. Prepare the container for warming the arteries in the incubation chamber by lining with KimWipes™ and adding a small amount of water to keep the arteries moist.
5. Check the tubing for air bubbles. If bubbles exist, turn on the pump and allow the 0.9% saline to flow until all bubbles are removed.

Application of the Dressing
1. Open the haemostatic dressing pouch and remove haemostatic dressing
2. Place the haemostatic dressing, mesh backing side UP, over the hole in the artery
3. Slowly wet the haemostatic dressing with an amount of saline appropriate for the article being tested
   NOTE: A standard (13-15 mg/cm² of fibrinogen) 2.4×2.4 cm haemostatic dressing should be wet with 800 μl of saline or other blood substitute. The amount of saline used can be adjusted depending on the requirements of the particular experiment being performed; however, any changes should be noted on the data collection forms.
   NOTE: Wet the haemostatic dressing drop wise with 0.9% saline warmed to 29-33° C. or other blood substitute, taking care to keep the saline from running off the edges. Any obvious differences in wetting characteristics from the positive control should be noted on data collection forms.
4. Place the shield gently onto the haemostatic dressing, taking care that it lies flat between the O-rings. Press lightly to secure in place
5. Wrap the artery and haemostatic dressing with plastic wrap
6. Wrap with blood pressure cuff, taking care that the bladder is adjacent to the haemostatic dressing.
7. Pump up the bladder to 100-120 mmHg, and monitor the pressure and pump again if it falls below 100 mmHg. Maintain pressure for 5 minutes.
   NOTE: Time and pressure can be altered according to the requirements of the experiment; changes from the standard conditions should be noted on the data collection forms.
8. After polymerization, carefully unwrap the artery and note the condition of the haemostatic dressing. Any variation from the positive control should be noted on the data collection form.
   EXCLUSION CRITERION: The mesh backing must remain over the hole in the artery. If it has shifted during the polymerization and does not completely cover the hole the haemostatic dressing must be excluded.

### Testing Procedure

Diagram of Testing Equipment Set-Up

The set-up of the testing equipment is shown in FIG. 2. Some additional, unshown components may be utilized to read out (pressure gauge) or control the pressure within the system

Equipment and Artery Assembly

Fill the artery and syringe with red 0.9% saline warmed to 37° C., taking care to minimize the amount of air bubbles within the syringe and artery. Filling the artery with the opening uppermost can assist with this. Attach the artery and syringe to the testing apparatus, making sure that there are as few air bubbles in the tubing as possible. The peristaltic pump should be calibrated so that it delivers approximately 3 ml/min. If available, the PLC should be operated according to a pre-determined range of pressures and hold times as appropriate for the article being tested. If under manual control, the pressure/time profile to be followed is attained by manually turning the pump on and off while referencing the system pressure as read out by one or more pressure-reading components of the system. Following the conclusion of testing, the haemostatic dressing is subjectively assessed with regard to adhesion to the artery and formation of a plug in the artery hole. Any variations from the positive control should be noted on the data collection form.

### Success Criteria

Haemostatic dressings that are able to withstand pressures for 3 minutes are considered to have passed the assay. When a haemostatic dressing has successfully passed the assay the data collection should be stopped immediately so that the natural decrease in pressure that occurs in the artery once the test is ended isn't included on the graphs. Should the operator fail to stop data collection, these points can be deleted from the data file to avoid confusing the natural pressure decay that occurs post-test with an actual dressing failure. The entire testing period from application of the haemostatic dressing to completion must fall within pre-established criteria. The maximum pressure reached should be recorded on the data collection form.
NOTE: Typical challenge is 250 mmHg for three minutes in one step, but that may be altered based on the article being tested. Changes from the standard procedure should be noted on the data collection forms.

### Failure Criteria

Haemostatic dressings that start leaking saline at any point during testing are considered to have failed the assay.
NOTE: Build failures that are caused by artery swelling can be ignored and the test continued or re-started (as long as the total testing time doesn't fall beyond the established limit).

When leakage does occur, the pressure should be allowed to fall ˜20 mmHg before data collection is stopped so that the failure is easily observed on the graphs. The pressures at which leakage occurred should be recorded on the data collection form. Should the data collection stop in the middle of the experiment due to equipment failure the data can be collected by hand at 5 second intervals until the end of the test or haemostatic dressing failure, whichever happens first. The data points should be recorded on the back of the data collection form, clearly labeled, and entered by hand into the data tables.

### Exclusion Criteria

If the total testing period exceeds the maximum allowed for that procedure, regardless of cause, results must be excluded. If there are leaks from collaterals that can't be fixed either by patching or finger pressure the results must be excluded. If the test fails because of leaks at the O-rings, the results must be excluded. If the mesh backing does not completely cover the hole in the artery, the results must be excluded.

### Adherence Performance Testing

Equipment and Supplies

Hemostat(s), Porcine artery and haemostatic dressing (usually after completion of the EVPA Assay although it does not need to be performed to do the Adherence Assay)

I. Preparation of the Artery+Dressing

After application of the dressing without completion of the EVPA Assay, the dressing is ready for the Adherence Assay and Weight Limit Test (if applicable). After application of the dressing and subsequent EVPA Analysis, the artery and syringe system is then disconnected slowly from the pump so that solution does not spray everywhere. The warmed, red saline solution from the EVPA Assay remains in the syringe until the Adherence Assay and Weight Limit Test (if applicable) is completed.

### Performance of the Adherence Assay

1. After preparation of the artery and dressing (with or without EVPA analysis), gently lift the corner of the mesh and attach a hemostat of known mass to the corner.
NOTE: If the FD developed a channel leak during the performance of the EVPA Assay, test the adherence on the opposite of the haemostatic dressing to obtain a more accurate assessment of the overall adherence.

2. Gently let go of the hemostat, taking care not to allow the hemostat to drop or twist. Turn the syringe so that the hemostat is near the top and allow the hemostat to peel back the dressing as far as the dressing will permit. This usually occurs within 10 seconds. After the hemostat has stopped peeling back the dressing, rate the adherence of the bandage according to the following scale:

| Dressing Performance Score | Amount of Adherence |
|---|---|
| 4 | 90+% |
| 3 | 75-90% |
| 2 | 50-75% |
| 1 | ~50% |
| 0.5 | Only the plug holds the hemostat |
| 0 | No adherence |

### Exclusion Criteria

The mesh backing must remain over the hole in the artery. If it has shifted during the polymerization and does not completely cover the hole the haemostatic dressing must be excluded.

### Success Criteria

Dressings that are given an adherence score of 3 are considered to have passed the assay.

### Failure Criteria

If a dressing does not adhere to the artery after application and/or prior to performing the EVPA assay, it is given a score of 0 and fails the adherence test. If a dressing receives a score≦2, the dressing is considered to have failed the Adherence Assay.

### Weight Held Performance Assay

After the initial scoring of the “Adherence Test”, weights may then be added to the hemostat in an incremental manner until the mesh backing is pulled entirely off of the artery. The maximum weight that the dressing holds is then recorded as a measure of the amount of weight the dressing could hold attached to the artery.

### Moisture Assay

Moisture determinations were carried out using a Brinkman Metrohm Moisture Analyzer System. The system contains the following individual components, 774 Oven Sample Processor, 774SC Controller, 836 Titrando, 5 ml and 50 ml 800 Dosino Units and a 801 Stirrer. The system was connected to a computer using the Brinkman Tiamo software for data collection, analysis and storage. The moisture system is set-up and run according to the manufactures recommendations and specifications to measure the moisture content of lyophilized samples using the Karl Fischer method.

All components were turned on and allowed to reach operating temperature prior to use. Lactose and water were run as standards and to calibrate the instrument. Once the machine was successfully calibrated, samples were prepared as follows. Dressing pieces weighing at least 30mg were placed into vials and capped. The vials were placed in the 774 Oven Sample Processor in numerical order, and one empty capped vial is placed in the conditioning space. The machine was then run to determine the moisture content (residual moisture) in the controls and samples.

### SDS-PAGE Gel Electrophoresis

Each dressing is cut into ¼'s, approximately 50 mg per section, and a section is then placed into a 15 mL conical tube. For the production control (ie Time 0), 1.0 mL of Okuda Dissolving Solution (10 M Urea, 0.1% Sodium Dodecyl Sulfate, 0.1% β-Mercaptoethanol) is added. For the remaining 3 pieces, 80 μL of 0.9% Saline is added to wet the dressing. The pieces are then incubated at 37° C. for 2, 5, and 10 minutes or such time as desired. To stop the reaction at the desired time, 1.0 mL of the Okuda Dissolving solution is added. The samples are then incubated at room temperature overnight, and then incubated at 70° C. for 30 minutes.

To prepare the samples for loading onto the gel, the samples which were previously dissolved in the Okuda Dissolving Solution were added to Sample buffer so that a 20 μL aliquot contains 10 μg. One μL of 0.1 M Dithiothreitol was then added to each sample. Twenty μL of each diluted sample is then loaded onto an 8% Tris-Glycine gel (Invitrogen), 1.0 mm thick, 10 wells. The gels were then run at 140V until the dye front reached the end of the gel. They were then removed and placed into Coomassie Blue Stain (50% v/v Methanol, 0.25% w/v Coomassie Brilliant Blue, 10% w/v Acetic Acid in ddH2O) on a shaking platform for a minimum of 1 hour. The gel is then transferred to the Destain Solution (25% Methanol, 10% Acetic Acid, 65% ddH2O) on a shaking platform until the background is nearly colorless.

After destaining, the gels were scanned, and the γ-γ dimer bands and the Aα, and Bβ bands analyzed by Scion densitometry software in order to determine the amount of conversion that occurred.

We claim:

1. A haemostatic composition comprising a frozen mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis.

2. The composition of claim 1, wherein the composition further comprises one or more foaming agents.

3. The composition of claim 1, wherein any of the proteinaceous components may originate in an animal species such as human, porcine, bovine, equine, caprine and piscine.

4. The composition of claim 1, wherein the composition further comprises one or more filler materials.

5. The composition of claim 1, wherein the composition further comprises one or more binding materials.

6. The composition of claim 1, wherein the compositions are substantially free of pathogens, such as bacteria, molds, spores, viruses and prions.

7. The composition of claim 1, wherein the composition further comprises one or more drugs or biologicals of therapeutic use.

8. The composition of claim 1, wherein the composition further comprises one or more solubilizing agents.

9. The composition of claim 1, wherein the composition further comprises one or more release agents.

10. A method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: (i) filling a suitable mold with the mixture components; and (ii) applying sufficient cooling to the mold and/or to the mixture components so as to freeze the mixture into a monolithic mass before excess fibrin formation occurs.

11. A haemostatic monolithic dressing for treating wounded tissue in a patient which comprises an effective mixture of dried fibrinogen and thrombin, with or without Factor XIII, which contains insufficient fibrin to prohibit its effective use as a haemostatic agent, and which further retains the ability to convert sufficient fibrinogen to fibrin, upon application to or mixing with a bodily fluid or an exogenous aqueous fluid and/or upon application to injured tissue, to provide effective hemostasis.

12. A method of treating wounded tissue, which comprises applying to said wounded tissue a monolithic haemostatic dressing according to claim 11.

13. A method for treating an injured patient comprising applying the haemostatic composition of claim 1 to an injured tissue in need of therapy.
